(19) United States
(12) Patent Application Publication
(10) Pub. No.: US 2020/0281711 A1
(43) Pub. Date: Sep. 10, 2020

(54) LONGITUDINALLY EXTENDABLE STENT GRAFT SYSTEMS AND METHODS

(71) Applicant: Endologix, Inc., Irvine, CA (US)

(72) Inventors: John McDermott, Coto de Caza, CA (US); Kaushik Patel, Poway, CA (US); Dale Ehnes, Forestville, CA (US)

(21) Appl. No.: 15/774,548

(22) PCT Filed: Jan. 23, 2018
(86) PCT No.: PCT/US2018/014920

§ 371 Date: May 8, 2018

Related U.S. Application Data
(60) Provisional application No. 62/453,460, filed on Feb. 1, 2017.

(51) Int. Cl. A61F 2/07; A61F 2/958

(52) U.S. Cl. CPC A61F 2/07; A61F 2/958; A61F 2220/0016; A61F 2002/065; A61F 2002/077; A61F 2210/0085; A61F 2250/0003; A61F 2250/0065

(57) ABSTRACT

A stent graft system includes a stent graft, a radially expandable scaffold, and an inflatable fill structure. The stent graft includes a body portion with a plurality of pleated sections that are configured to be extended from a telescopically compressed state to a longitudinally extended state. The radially expandable scaffold is attached to a top of the body portion, and has one or more fixation elements for penetrating into an aortic wall. The inflatable fill structure is attached at a top of the body portion and is configured to expand in a longitudinal direction as the body portion is extended in the longitudinal direction. The inflatable fill structure is also configured to expand in a radial direction to contact an inner surface of a blood vessel.

### CROSS REFERENCE TO RELATED APPLICATIONS

This application claims priority from U.S. Provisional Application Ser. No. 62/453,460, filed on Feb. 1, 2017, the entire contents of which are incorporated by reference herein.

### FIELD

Various embodiments in the present disclosure relate to stent grafts, systems including stent grafts, and methods of using such systems having stent grafts for treating aneurysms.

### BACKGROUND

Aneurysms are enlargements or bulges in blood vessels that are often prone to rupture and which therefore present a serious risk to a patient. Aneurysms may occur in any blood vessel but are of particular concern when they occur in the cerebral vasculature or the patient's aorta.

Abdominal aortic aneurysms (AAA's) are classified based on their location within the aorta as well as their shape and complexity. Aneurysms that are found below the renal arteries are referred to as infrarenal abdominal aortic aneurysms. Suprarenal abdominal aortic aneurysms occur above the renal arteries. Thoracic aortic aneurysms (TAA's) occur in the ascending, transverse, or descending part of the upper aorta. Infrarenal aneurysms are the most common, representing about 70% of all aortic aneurysms. Suprarenal aneurysms are less common, representing about 20% of the aortic aneurysms. Thoracic aortic aneurysms are the least common and often the most difficult to treat.

The most common form of aneurysm is “fusiform,” where the enlargement extends about the entire aortic circumference. Less commonly, the aneurysms may be characterized by a bulge on one side of the blood vessel attached at a narrow neck. Thoracic aortic aneurysms are often dissecting aneurysms caused by hemorrhagic separation in the aortic wall, usually within the medial layer. A common treatment for each of these types and forms of aneurysm is open surgical repair. Open surgical repair is often quite successful in patients who are otherwise reasonably healthy and free from significant co-morbidities. Such open surgical procedures are problematic, however, since access to the abdominal and thoracic aortas is difficult to obtain and because the aorta must be clamped off, placing significant strain on the patient's heart.

Endoluminal gratis have come into widespread use for the treatment of aortic aneurysms in patients. In general, endoluminal repairs access the aneurysm “endoluminally” through either or both common iliac arteries. The grafts are then implanted. Successful endoluminal procedures have a much shorter recovery period than open surgical procedures.

### SUMMARY OF THE DISCLOSURE

One or more aspects of example embodiments are directed to stent grafts, stent graft systems, and methods of using stent graft systems. According to an example embodiment, a stent graft system includes a stent graft, a radially expandable scaffold, and an inflatable fill structure. Ira various embodiments, the stent graft includes a body portion with a plurality of pleated sections that are configured to be extended from a telescopically compressed state to a longitudinally extended state. In various embodiments, the radially expandable scaffold is attached to a top of the body portion of the stent graft, and has one or more fixation elements for penetrating into an aortic wall. In various embodiments, the inflatable fill structure is attached at a top of the body portion of the stent graft and is configured to expand in a longitudinal direction as the body portion of the stent graft is extended in the longitudinal direction.

In various embodiments, the inflatable fill structure is not attached at or to a central part or middle part of the body portion of the stent graft. In various embodiments, the inflatable fill structure is further attached at a lower part of the body portion of the stent graft. In various embodiments, an amount that the inflatable fill structure expands in the longitudinal direction corresponds to an amount that the body portion is extended in the longitudinal direction.

In some embodiments, the inflatable structure includes an inner wall adjacent to an outer surface of the body portion, and also includes an outer wall. In some embodiments, the inner wall is configured to contact the outer surface of the body portion when the inflatable fill structure is inflated to provide columnar support to the body portion. In various embodiments, the outer wall is configured to conform to an inner surface of a vessel in which the stent graft is inserted.

In various embodiments, the stent graft further includes a first leg portion, a second leg portion, and a transition portion connecting the first leg portion and the second leg portion to the body portion. In various embodiments, at least one of the first leg portion and the second leg portion is configured to be extendable from a telescopically compressed state to a longitudinally extended state.

In various embodiments, a length of the body portion in the telescopically compressed state is less than one-fourth of a length of the body portion in the longitudinally extended state. In various embodiments, a length of the body portion in the telescopically compressed state is less than one-half of a length of the body portion in the longitudinally extended state.

A method in accordance with various embodiments for deploying a stent graft system to repair an aneurysm includes inserting, into an aorta, the stent graft system with a body portion of the stent graft system in a telescopically compressed state, longitudinally extending the body portion of the stent graft system from the telescopically compressed state to a longitudinally extended state, and filling an inflatable fill structure surrounding at least a portion of the body portion to provide columnar support for the body portion.

In various embodiments, the inflatable fill structure is attached to at least a top of the body portion. In various embodiments, the inflatable fill structure is not attached at a central part of the body portion. In various embodiments, the inflatable fill structure expands or extends in a longitudinal direction as the body portion is extended in the longitudinal direction. in various embodiments, an amount that the inflatable fill structure expands in the longitudinal direction corresponds to an amount that the body portion extends in the longitudinal direction.

In various embodiments, the longitudinally extending of the body portion includes pulling a first leg portion connected to a lower part of the body portion into an iliac artery and pulling a second leg portion connected to a lower part of the body portion into another iliac artery, In various embodiments, the filling of the inflatable fill structure includes expanding the inflatable fill structure in a longitudinal direction as the body portion is extended in the longitudinal direction, filling the inflatable fill structure with saline to expand the inflatable fill structure in a radial direction, evacuating the saline from the inflatable fill structure, and filling the inflatable fill structure with a hardenable fill medium.

In various embodiments, the hardenable fill medium includes a polymer such as a liquid polymer that hardens as it is dried or cured. In various embodiments, the inflatable fill structure is radially expanded to conform to an inner surface of the aorta after being longitudinally extended along with the body portion. In various embodiments, the method further includes longitudinally extending a first leg portion of the stent graft system from a telescopically compressed state to a longitudinally extended state, and longitudinally extending a second leg portion of the stent graft system from a telescopically compressed state to a longitudinally extended state, where the first leg portion and the second leg portion are connected to the body portion of the stent graft system.

A method in accordance with various embodiments for repairing an aneurysm includes inserting, into an aorta, a stent graft system. In various embodiments, the stent graft system includes a body portion that is configured to be extended from a telescopically compressed state to a longitudinally extended state, an inflatable fill structure surrounding the body portion, a first leg portion connected to the body portion, and a second leg portion connected to the body portion. In various embodiments, the method further includes longitudinally extending the body portion of the stem graft system from the telescopically compressed state to the longitudinally extended state by pulling the first leg portion into an iliac artery and pulling the second leg portion into another iliac artery. In various embodiments, the method also includes filling the inflatable fill structure so that the inflatable fill structure radially expands to conform to an inner surface of the aorta and provides columnar support for the body portion.

A stent graft system in accordance with an embodiment includes a stent graft, a radially expandable scaffold, and an inflatable fill structure. The stent graft includes a body portion with a plurality of pleated sections that are configured to be extended from a telescopically compressed state to a longitudinally extended state. The radially expandable scaffold is attached to a top of the body portion and has one or more fixation elements for penetrating into an aortic wall. The inflatable fill structure is positioned at top section of the body portion and is configured to not expand in a longitudinal direction as the body portion is extended in the longitudinal direction. In various embodiments, the inflatable fill structure is configured to provide a seal at a proximal neck of an aneurysm.

### BRIEF DESCRIPTION OF THE DRAWINGS

FIG. 1 is an illustration of a cross section of an example anatomy with an infrarenal aortic aneurysm;

FIG. 2 is an illustration of a stent graft in accordance with an embodiment in a longitudinally extended state;

FIG. 3 is an illustration of the stent graft of FIG. 2 in accordance with an embodiment in a compressed state;

FIG. 4 is an illustration of a stent graft in accordance with another embodiment in a compressed state;

FIG. 5 is an illustration of a stent graft system in accordance with an embodiment in a longitudinally extended state;

FIG. 6 is an illustration of the stent graft system of FIG. 5 in a longitudinally extended state in an aorta and iliac arteries;

FIG. 7 is an illustration of the stent graft system of FIG. 5 in accordance with an embodiment in a compressed state in an aorta and fixed to the aorta at a proximal end of the stent graft system;

FIG. 8 is an illustration of the stent graft system of FIG. 5 in accordance with an embodiment in a compressed state in an aorta and iliac arteries and placed over an aortic bifurcation;

FIG. 9 is an illustration of the stent graft system of FIG. 6 in accordance with an embodiment after the inflatable fill structure has been filled with a hardenable fill medium;

FIGS. 10A, 10B, 10C, 10D, 10E, and 10F are illustrations showing some steps during placement, extension, and filling of the stent graft system of FIG. 5 in an aneurysm, in accordance with various illustrative embodiments;

FIG. 11 is a flow diagram illustrating a method of using the stent graft system of FIGS. 10A, 10B, 10C, and 10D, in accordance with an illustrative embodiment;

FIG. 12 is an illustration of a stern graft system in accordance with another embodiment in a compressed state;

FIG. 13 is an illustration of the stent graft system of FIG. 12 in a longitudinally extended state;

FIGS. 14A, 14B, 14C, and 14D illustrate flowcharts of methods in accordance with various embodiments; and

FIG. 15 is an illustration of a stent graft system in accordance with another embodiment in an aorta.

### DETAILED DESCRIPTION

Hereinafter, example embodiments will be described in more detail with reference to the accompanying drawings. The present invention, however, may be embodied in various different forms, and should not be construed as being limited to only the illustrated embodiments herein. Rather, these embodiments are provided as examples so that this disclosure will be thorough and complete, and will fully convey the aspects and features of the present disclosure to those skilled in the art. Unless otherwise noted, like reference numerals denote like elements throughout the attached drawings and the written description, and thus, descriptions thereof may not be repeated.

It will be understood that the aspects and features of the present disclosure, as generally described herein, and illustrated in the figures, can be arranged, substituted, combined, and designed in a wide variety of different configurations, all of which are explicitly contemplated and make part of this disclosure. Accordingly, descriptions of features or aspects within each example embodiment should typically be considered as available for other similar features or aspects in other example embodiments.

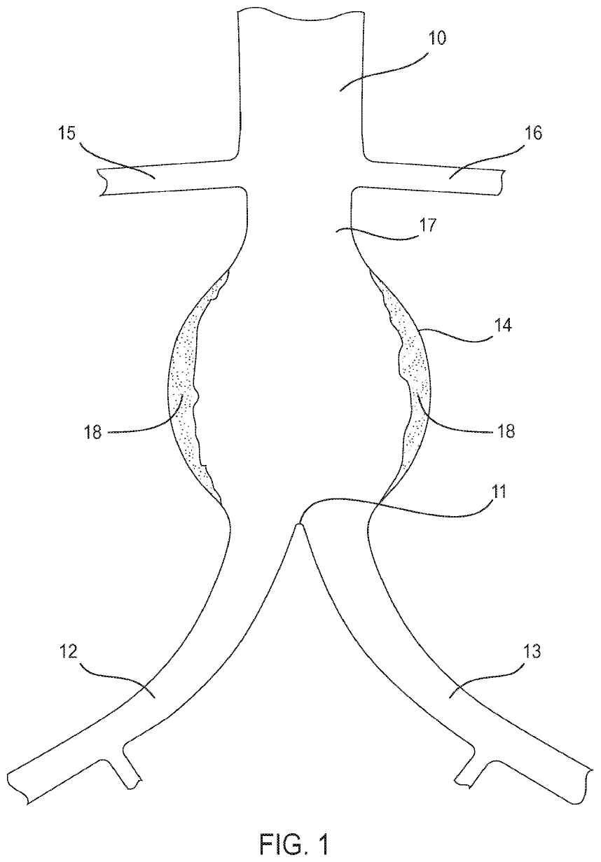

FIG. 1 is an illustration of a cross section of an example anatomy with an infrarenal aortic aneurysm. In FIG. 1, an aorta 10 branches at an aortic bifurcation 11 into two iliac arteries 12 and 13. An aneurysm sac 14 denotes a bulged section of the aorta 10. As the name implies, the infrarenal aortic aneurysm is located below renal arteries 15 and 16. A segment of the aorta 10 between the renal arteries 15 and 16 and the aneurysm sac 14 is referred to as a proximal neck 17. Often mural thrombus 18 forms on an inside wall of the aneurysm sac 14. A diameter of a flow lumen in the aneurysm is, thus, reduced by the mural thrombus 18 to a diameter less than a diameter of the aneurysm sac 14.

The dimensions of an aortic aneurysm can vary greatly from patient to patient. The diameter of the proximal neck 17 may vary, for example, from 18 millimeters (mm) to 34 mm. The distance from the aortic bifurcation 11 to the renal arteries 15 and 16 may vary, for example, from 80 mm to 160 mm. The diameters of the iliac arteries 12 and 13 might not be the same as each other. The diameters of the iliac arteries 12 and 13 may vary, for example, from 8 mm to 20 mm, One iliac artery or both iliac arteries 12 and 13 may be aneurysmal with greatly enlarged diameters, for example, of more than 30 mm.

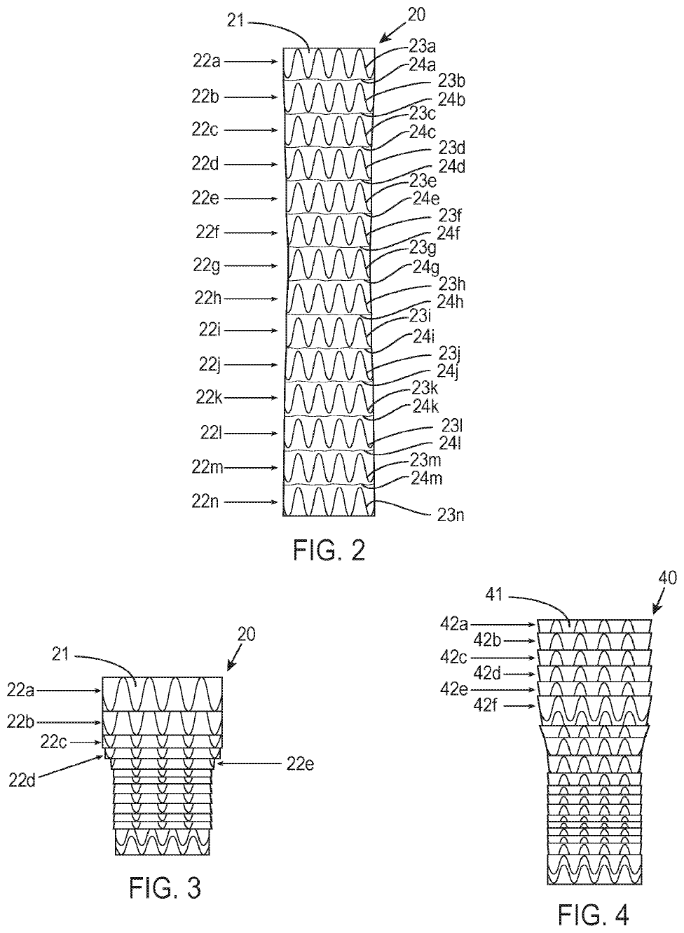

FIG. 2 illustrates an embodiment of a stent graft 20 in accordance with an embodiment in a longitudinally extended state. The stent graft 20 includes a graft 21. In various embodiments, the graft 21 is made of a polymer. In some embodiments, the graft 21 is made of expanded Polytetrafluoroethylene (ePTFE). The stent graft 20 includes pleated sections 22a, 22b, 22c, 22d, 22e, 22f, 22g, 22h, 22i, 22j, 22k, 22l, 22m, and 22n. Each of the pleated sections 22a, 22b, 22c, 22d, 22e, 22f, 22g, 22h, 22i, 22j, 22k, 22l, 22m, and 22n includes a scaffold 23a, 23b, 23c, 23d, 23e, 23f, 23g, 23h, 23i, 23j, 23k, 23l, 23m, and 23n, respectively, that is encapsulated within or attached to a corresponding portion of the graft 21. The scaffolds 23a, 23b, 23c, 23d, 23e, 23f, 23g, 23h, 23i, 23j, 23k, 23l, 23m, and 23n may each comprise, for example, a sinusoidal stent frame in a ring shape and may be made from, for example, cobalt-chromium (CoCr) alloy, stainless steel, nitinol, or the like. Each of the scaffolds 23a, 23b, 23c, 23d, 23e, 23f, 23g, 23h, 23i, 23j, 23k, 23l, 23m, and 23n is radially expandable. When longitudinally extended, the graft 21 is a generally tubular shape that allows for blood flow through a lumen within the graft 21.

Each of the pleated sections 22a, 22b, 22c, 22d, 22e, 22f, 22g, 22h, 22i, ??j, 22k, 22l, and 22m ends with a pleat 24a, 24b, 24c, 24d, 24e, 24f, 24g, 24h, 24i, 24j, 24k, 24l, and 24m, respectively, in the graft 21. The pleats 24a, 24b, 24c, 24d, 24e, 24l, 24g, 24h, 24i, 24j, 24k, 24l, and 24m in the graft 21 allow for the graft 21 to fold in at those locations, so that the stent graft 20 can be telescoping to be placed in a compressed state, and to allow for movement from the compressed state to the longitudinally extended state.

FIG. 3 illustrates an embodiment of the stent graft 20 in accordance with an embodiment in the compressed state. The graft 21 is pleated such that the pleated section 22b can fold at least partially up under the pleated section 22a, the pleated section 22c can fold at least partially up under the pleated section 22b, the pleated section 22d can fold at least partially up under the pleated section 22c, the pleated section 22e can fold at least partially up under the pleated section 22d, and so on for each adjacent pleated section. In the compressed state, each of the pleated sections may have a slightly smaller diameter than the above adjacent pleated section so that they can fit within each other in a telescoping manner, and when the pleated section is extended (e.g., fully or partially) in the longitudinal direction, its diameter may expand radially. The stent graft 20 in the compressed state in FIG. 3 can be extended in the longitudinal direction until fully extended as shown in FIG. 2. In various embodiments, a length of the stent graft 20 in the compressed state may be less than one-fourth of the length of the stent graft 20 in the extended state. In some embodiments, a length of the stent graft 20 in the compressed state may be less than one-half of the length of the stern graft 20 in the extended state.

The pleating direction of the pleats 24a, 24b, 24c, 24d, 24e, 24f, 24g, 24h, 24i, 24j, 24k, 24l, and 24m in the embodiment of FIGS. 2 and 3 is formed such that the crown valleys are tucked abluminally in the compressed state. FIG. 4 shows another embodiment of a stent graft 40 in a compressed state that includes a graft 41 and pleated sections 12a, 42b, 42c, 42d, 42e, 42f, and so on. Pleats for the pleated sections in the embodiment of FIG. 4 are formed such that the crown and valleys are tucked adluminally. In various other embodiments, the pleats may be formed such that crown valleys are tucked adluminally on one end (e.g., a proximal end) of the stent graft and are tucked abluminally on an opposite end (e.g., a distal end) of the stent graft.

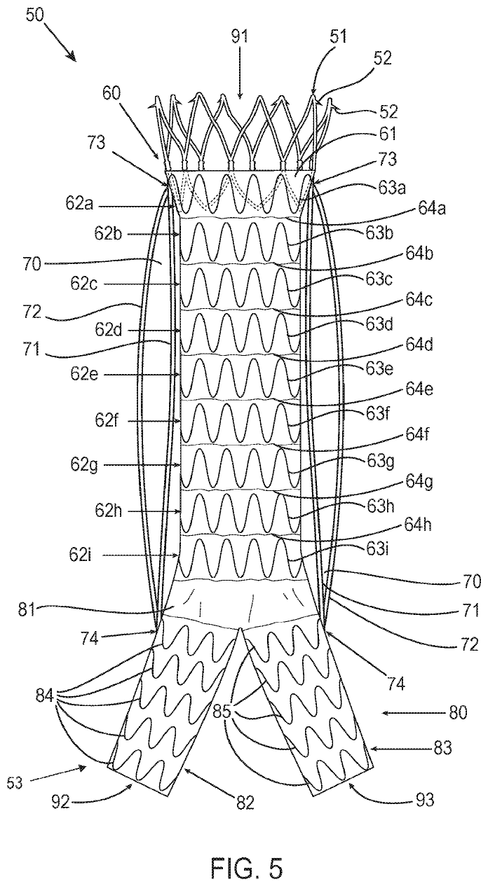

FIG. 5 illustrates an embodiment of a stent graft system 50 in accordance with an embodiment in a longitudinally extended state. The stent graft system 50 includes a stent graft 53 with a body portion 60 and a bifurcation portion 80. The bifurcation portion 80 includes a transition portion 81, a first leg portion 82, and a second leg portion 83. The stent graft system 50 includes a graft 61. In various embodiments, the graft 61 is used for the body portion 60 and the bifurcation portion 80. In some embodiments, one or more other grafts may be used for each of the portions of the stent graft system 50. In various embodiments, the graft 61 is made of a polymer. In some embodiments, the graft 61 is made of expanded Polytetratluoroethylene (ePTFE). The body portion 60 includes pleated sections 62a, 62b, 62c, 62d, 62e, 62f, 62g, 62h, and 62i. Each of the pleated sections 62a, 62b, 62c, 62d, 62e, 62f, 62g, 62h, and 62i includes a scaffold 63a, 63b, 63c, 63d, 63e, 63f, 63g, 6311, and 63i, respectively, that is encapsulated within or attached to a corresponding portion of the graft 61. The scaffolds 63a, 63b, 63c, 63d, 63e, 63f, 63g, 63h, and 63i may each comprise, for example, a sinusoidal stent frame in a ring shape and may be made from, for example, cobalt-chromium (CoCr) alloy, stainless steel, nitinol, or the like. Each of the scaffolds 63a, 63b, 63c, 63d, 63e, 63f, 63g, 63h, and 63i is radially expandable.

Each of the pleated sections 62a, 62b, 62c, 62d, 62e, 62f, 62g, and 62h ends with a pleat 64a, 64b, 64c, 64d, 64e, 64f, 64g, and 64h, respectively, in the graft 61. The pleats 64a, 64b, 64c, 64d, 64e, 64f, 64g, and 64h in the graft 61 allow for the graft 61 to fold in at those locations, so that the body portion 60 can be telescoping to be in a compressed state, and to allow for movement from the compressed state to the longitudinally extended state. In various embodiments, a length of the body portion 60 in the compressed state may be less than one-fourth of the length of the body portion 60 in the extended state. In various embodiments, a length of the body portion 60 in the compressed state may be less than one-half of the length of the body portion 60 in the extended state. When extended, the portion of the graft 61 for the body portion 60 is a generally tubular shape that allows for blood flow through a lumen within the body portion 60. The stent graft system 50 includes a radially expandable scaffold 51 connected to the top of the body portion 60 for providing fixation of the stent graft system 50 in an aorta along an aortic wall. The radially expandable scaffold 51 may, for example, have hooks or barbs 52 for penetrating into an aortic wall and thereby enhancing fixation.

The transition portion 81 includes a portion of the graft 61 that extends from the body portion 60 to each of the first leg portion 82 and the second leg portion 83. The transition portion 81 may be made of the same or different material from that of the graft 61. For example, in various embodiments, the transition portion 81 may be made of, for example, Teflon. The first leg portion 82 includes scaffolds 84 that are encapsulated within or attached to a corresponding portion of the graft 61. The scaffolds 84 may each comprise, for example, a sinusoidal stent frame in a ring shape and may be made from, for example, cobalt-chromium (CoCr) alloy, stainless steel, nitinol, or the like. Each of the scaffolds 84 is radially expandable. The second leg portion 83 includes scaffolds 85 that are encapsulated within or attached to a corresponding portion of the graft 61. The scaffolds 85 may each comprise, for example, a sinusoidal stent frame in a ring shape and may be made from, for example, cobalt-chromium (CoCr) alloy, stainless steel, nitinol, or the like. Each of the scaffolds 85 is radially expandable. Each of the first leg portion 82 and the second leg portion 83 is a generally tubular shape that allows for blood flow through a lumen within the first leg portion 82 and the second leg portion 83, respectively. The stent graft system 50 has a proximal end 91 for receiving blood flow and distal ends 92 and 93 out of which the blood is able to flow.

The stent graft system 50 includes an inflatable fill structure 70. The inflatable fill structure 70 may surround (e.g., entirely surround) the outer circumference of the body portion 60, and may be a single inflatable fill structure or a plurality of inflatable fill structures arranged around the body portion 60. The inflatable fill structure 70 includes an inner wall 71 and an outer wall 72. In various embodiments, the inflatable fill structure 70 is an endobag or the like. The inflatable fill structure 70 is attached at locations 73 near the top of the body portion 60 and is attached at locations 74 near the top of the leg portions 82 and 83. In various embodiments, the inflatable fill structure 70 is attached at the locations 73 and 74 and remains unattached from a middle part or central part of the body portion 60 to allow for the body portion 60 to be longitudinally extended. In various embodiments, the inflatable fill structure 70 is stitched to the graft 61 at the locations 73 and 74. In various embodiments, the inflatable fill structure 70 is fillable through a fill tube with a hardenable filling material such as Polyethylene glycol (PEG) or another polymer that may be polymerized in situ.

According to various embodiments, the inflatable fill structure 70 is highly stretchable (e.g., up to 5000% from an initial state) to conform to each of the compressed state and the longitudinally extended state of the body portion 60, while having reduced retraction forces and packing densities than those of other till structures. For example, the inflatable fill structure 70 may have low flexural modulus, providing expansion (e.g., longitudinally and radially) at low pressures (e.g., 3 to 100 mm Hg, or more desirably, 3 to 5 mm Hg). Thus, in various embodiments, when the body portion 60 is in a compressed state (e.g., a fully compressed state), a length of the inflatable fill structure 70 in the longitudinal direction may be at its shortest. For example, when the body portion 60 is in a compressed state (e,g., a fully compressed state), the inflatable fill structure 70 may be in an initial state (e.g., relaxed or unstretched state). As the body portion 60 is extended in the longitudinal direction, the inflatable fill structure 70 is extended or expanded (e.g., stretched or pulled) in the longitudinal direction, so that the length of the inflatable fill structure 70 increases in the longitudinal direction according to an amount that the body portion 60 is extended.

According to various embodiments, the inflatable fill structure 70 may be made of, for example, polyurethane, silicon., Teflon, and/or combinations thereof. The polyurethane may include, for example, Pellethane® 5863 80A or softer, and/or Estane® 58123 70A or the like. The silicone may include any from among NuSil's MED-4714, MED-4720, MED-4810, MED-4820, combinations thereof, or the like. Accordingly, the inflatable fill structure 70 may be thinner and more elastic than that of other fill structures. In various embodiments, the inflatable fill structure 70 is made of an aromatic polyether-based thermoplastic polyurethane (TPU). In various embodiments, the inflatable fill structure 70 is made of silicone rubber or a silicone elastomer. In some embodiments, the Shore durometer hardness of the inflatable fill structure 70 is 80A or softer. In some embodiments, the Shore durometer hardness of the inflatable fill structure 70 is within a range of 10A to 80A. In various embodiments, the ultimate elongation of the material for the inflatable fill structure 70 is within a range of 700% to 1,400%.

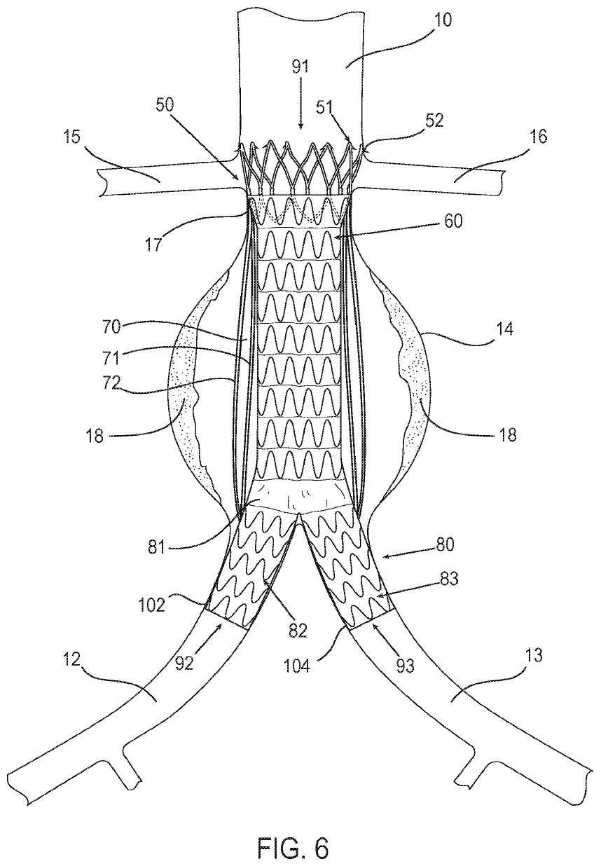

FIG. 6 is an illustration of the stent graft system 50 in accordance with an embodiment in a longitudinally extended state in the aorta 10 and the iliac arteries 12. and 13. In various embodiments, the body portion 60 of the stent graft system 50 is able to be extended and/or expand across the aneurysm sac 14 to exclude the aneurysm sac 14 from aortic blood pressure. The stent graft system 50 includes the body portion 60 that can be placed in the aorta 10, and first and second leg portions 82 and 83 extending from the body portion 60 that can be placed into the iliac arteries 12 and 13, respectively. A proximal end of the body portion 60 can radially expand against a wall of the aorta 10 at the proximal neck 17 to create a proximal seal. Distal ends 92. and 93 of the first and second leg portions 82 and 83 can radially expand against walls of the iliac arteries 12 and 13, respectively, to form distal seals in the distal seal zones 102 and 104 adjacent thereto. The barbs 52 of the radially expandable scaffold 51 can penetrate into the wall of the aorta 10, thereby enhancing fixation of the stent graft system 50. Blood is able to flow from the proximal end 91 through the body portion 60 and out of the distal ends 92 and 93 of the first and second leg portions 82 and 83, respectively. The inflatable fill structure 70 is initially in an uninflated state, but may be expanded or extended (e.g., stretched or pulled) in the longitudinal direction according to the amount of extension of the body portion 60.

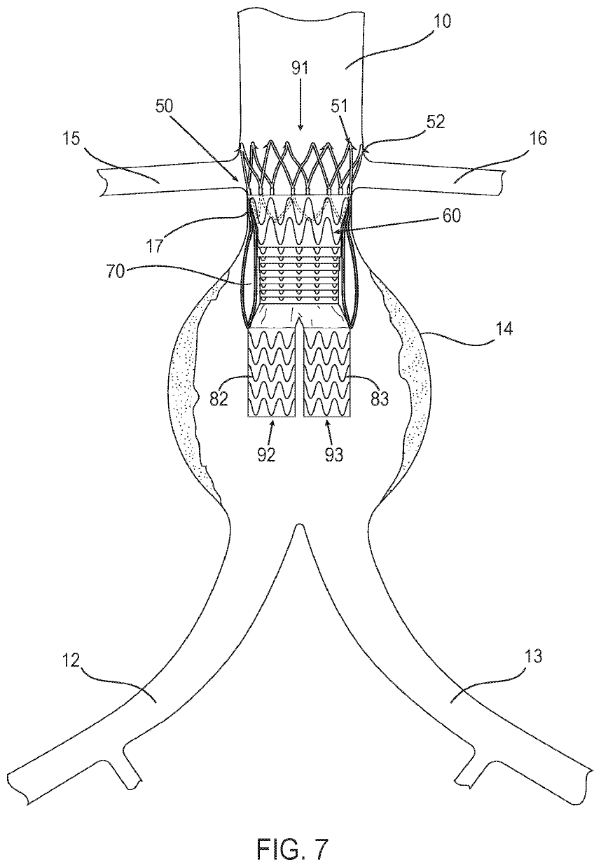

FIG. 7 is an illustration of the stem graft system 50 in accordance with an embodiment in a compressed state in the aorta 10 and fixed to the aorta 10 at a proximal end of the stent graft system 50. In various embodiments, the stent graft system 50 is initially inserted into the aorta 10 in the compressed state in which the body portion 60 of the stent graft system 50 is compressed, and the inflatable fill structure 70 is in the initial state e.g., relaxed or unstretched state) as shown in FIG. 7. With reference to FIGS. 5 and 7, the portion of the graft 61 for the body portion 60 is pleated such that the pleated section 62b can fold at least partially up under the pleated section 62a, the pleated section 62c can fold at least partially up under the pleated section 62b, the pleated section 62d can fold at least partially up under the pleated section 62c, the pleated section 62e can fold at least partially up under the pleated section 62d, the pleated section 62f can fold at least partially up under the pleated section 62e, the pleated section 62g can fold at least partially up under the pleated section 62f, the pleated section 62h can fold at least partially up under the pleated section 62g, and the pleated section 62i can fold at least partially up under the pleated section 62h. FIG. 7 shows the body portion 60 in a telescopically compressed state and the body portion 60 is configured to be extendable from the compressed state of FIG. 7 to the longitudinally extended state of FIG. 6, In the compressed state, each of the pleated sections of the body portion 60 may have a slightly smaller diameter than the above adjacent pleated section so that they can fit within each other in a telescoping manner, In the expanded state, each of the pleated sections of the body portion 60 may be radially expanded. In the expanded state, each of the pleated sections may have the same or substantially the same diameter as those of adjacent pleated sections. However, the present invention is not limited thereto, for example, at least one of the pleated sections of the body portion 60 may have a smaller diameter even in the expanded state than that of an adjacent pleated section.

With reference to FIGS. 6 and 7, the inflatable fill structure 70 is sized and configured such that the inflatable fill structure 70 is able to longitudinally extend or expand as the body portion 60 is longitudinally extended. For example, the inflatable fill structure 70 may be expanded or extended (e.g., stretched or pulled) in the longitudinal direction according to the amount of extension of the body portion 60. The barbs 52 of the radially expandable scaffold 51 can penetrate into the wall of the aorta 10, thereby enhancing fixation of the stem graft system 50. In various embodiments, the distal ends 92 and 93 of the first and second leg portions 82 and 83, respectively, are pulled to cause the body portion 60 to longitudinally extend from the telescoped compressed state to the longitudinally extended state, and to place the first and second leg portions 82 and 83 in the iliac arteries 12 and 13, respectively.

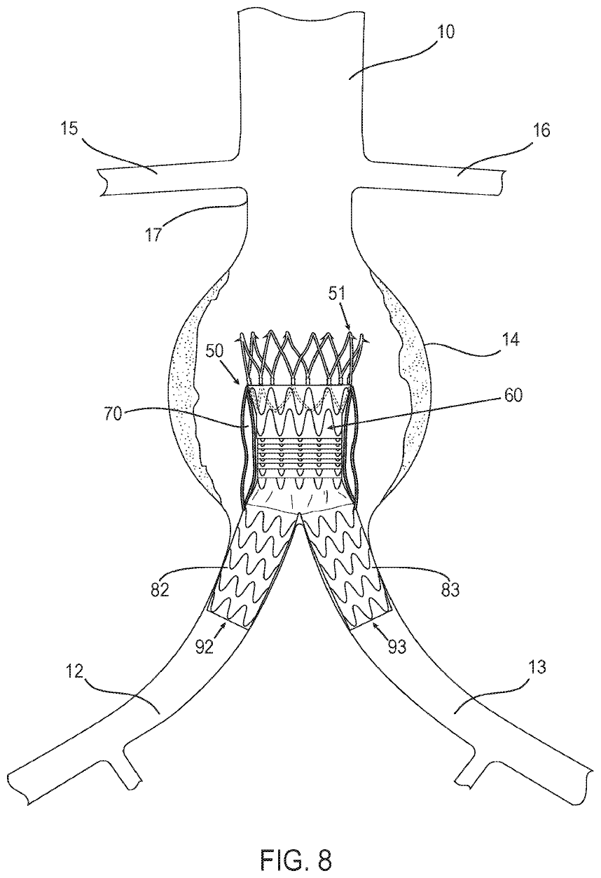

FIG. 8 is an illustration of the stent graft system 50 in accordance with an embodiment in a compressed state in the aorta 10 and iliac arteries 12 and 13 and located over the aortic bifurcation. In various embodiments, the stent graft system 50 is initially inserted into the aorta 10 in the compressed state in which the body portion 60 of the stent graft system 50 is compressed as shown in FIG. 8. With reference to FIGS. 5 and 8, the portion of the graft 61 for the body portion 60 is pleated such that the pleated section 62b can fold at least partially up under the pleated section 62a, the pleated section 62c can fold at least partially up under the pleated section 62b, the pleated section 62d can fold at least partially up under the pleated section 62c, the pleated section 62e can fold at least partially up under the pleated section 62d, the pleated section 62f can fold at least partially up under the pleated section 62e, the pleated section 62g can fold at least partially up under the pleated section 62f, the pleated section 62h can fold at least partially up under the pleated section 62g, the pleated section 62i can fold at least partially up under the pleated section 62h. FIG. 8 shows the body portion 60 in a telescopically compressed state and the body portion 60 is configured to be extendable from the compressed state of FIG. 8 to the longitudinally extended state of FIG. 6. In the compressed state, each of the pleated sections of the body portion 60 may have a slightly smaller diameter than the above adjacent pleated section so that they can fit within each other in a telescoping manner. In the expanded state, each of the pleated sections of the body portion 60 may be radially expanded. In the expanded state, each of the pleated sections may have the same or substantially the same diameter as those of adjacent pleated sections. However, the present invention is not limited thereto, for example, at least one of the pleated sections of the body portion 60 may have a smaller diameter even in the expanded state than that of an adjacent pleated section.

With reference to FIGS. 6 and 8, the inflatable fill structure 70 is sized and configured such that the inflatable fill structure 70 is able to longitudinally extend or expand as the body portion 60 is longitudinally extended. For example, the inflatable fill structure 70 may be expanded or extended (e.g., stretched or pulled) in the longitudinal direction according to the amount of extension of the body portion 60. The distal ends 92 and 93 of the first and second leg portions 82 and 83 are positioned in the iliac arteries 12 and 13, respectively. In various embodiments, the radially expandable scaffold 51 can be pulled up to cause the body portion 60 to longitudinally extend from the telescoped compressed state to the longitudinally extended state, and to place the radially expandable scaffold 51 above the aneurysm sac 14 as shown in FIG. 6.

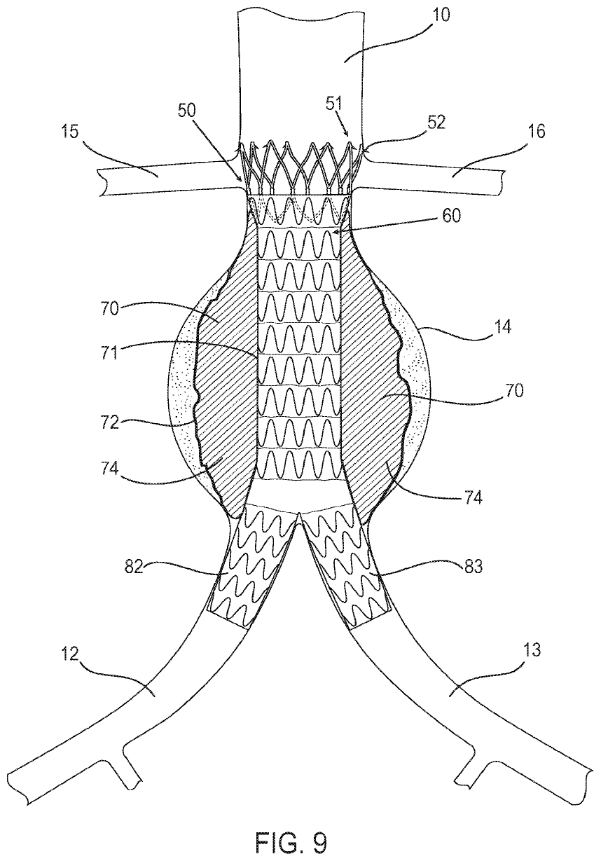

FIG. 9 is an illustration of the stent graft system 50 in accordance with an embodiment after the inflatable fill structure 70 has been filled with a hardenable filling medium. According to various embodiments, the hardenable filling medium may include, for example, a liquid polymer that hardens as it is dried or cured, such as a hydrogel or the like. However, the present invention is not limited thereto, and any suitable hardenable filling medium may be used to fill the inflatable fill structure 70.

Referring to FIG. 9, in various embodiments, when the inflatable fill structure 70 is inflated or filled, the inner wall 71 of the inflatable fill structure 70 conforms to an outer surface of the body portion 60 down to a portion of an outer surface of the first and second leg portions 82 and 83, Accordingly, the inflatable fill structure 70 may provide columnar support for the body portion 60 when inflated or filled. Also, when the inflatable fill structure 70 is inflated or filled as shown in the embodiment of FIG. 9, the outer wall 72 of the inflatable fill structure 70 expands radially to conform to an inner wall of the aneurysm sac 14 to aid in preventing leaks of blood into the aneurysm sac 14.

With reference to FIGS. 6, 7, 8, and 9, by having the body portion 60 be longitudinally extendable and the inflatable fill structure 70 be highly conformable, the body portion 60 can be extended to different lengths and the inflatable fill structure 70 can be conformed to different volumes depending on the dimensions of the aneurysm being treated, which allows for treating a wide range of patients. In such a manner, stent graft systems having a design of the stent graft system 50 can be used in patients with differing aneurysm lengths and volumes, and the body portion 60 can be extended to a length that suits the dimensions of the patient, while the inflatable fill structure 70 can be inflated or filled to a volume that suits the dimensions of the patient.

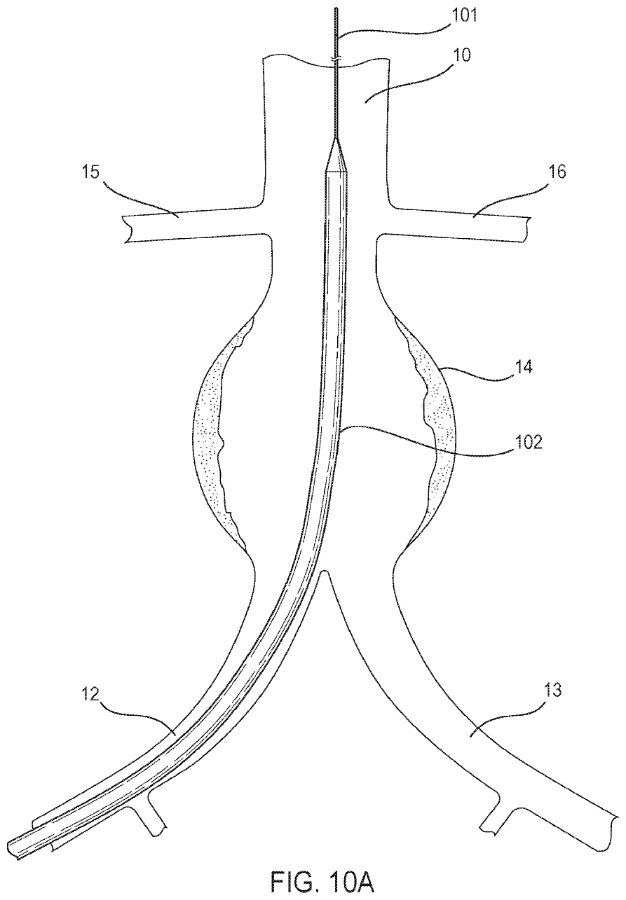

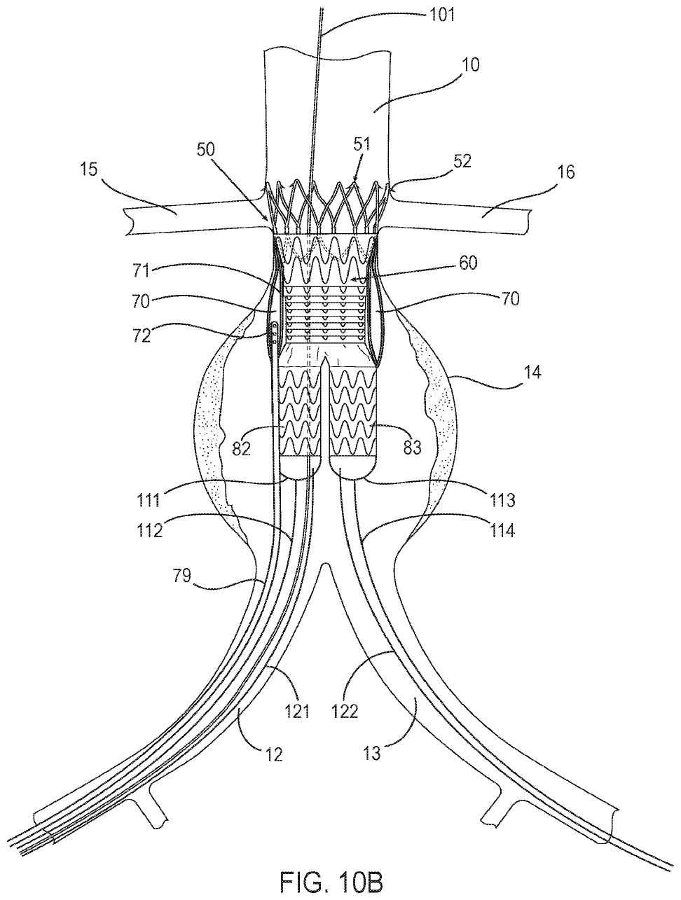

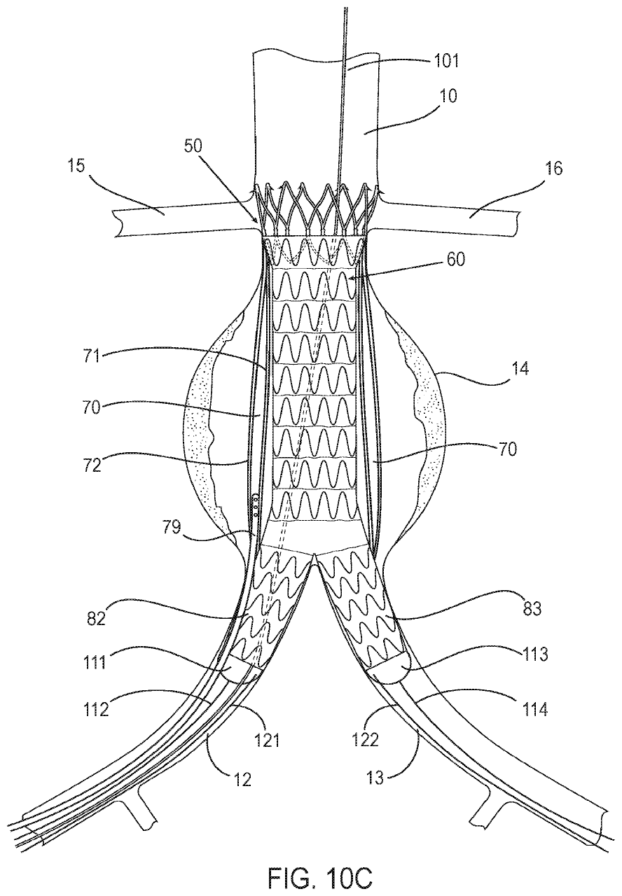

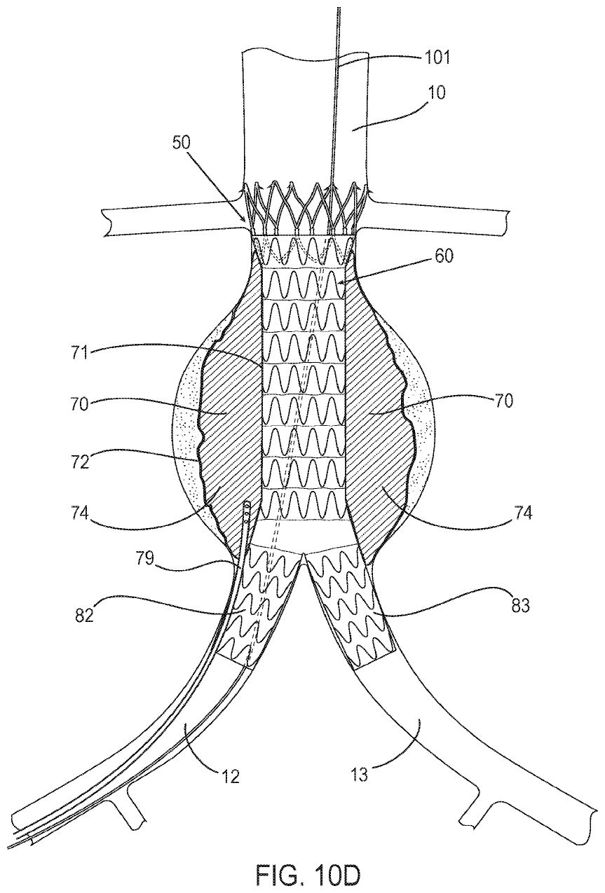

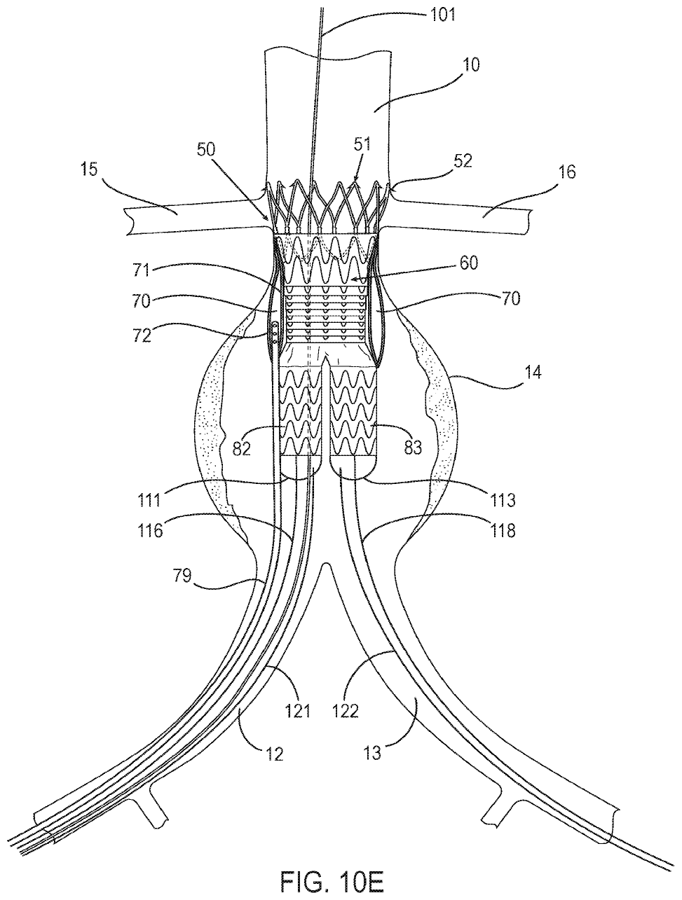

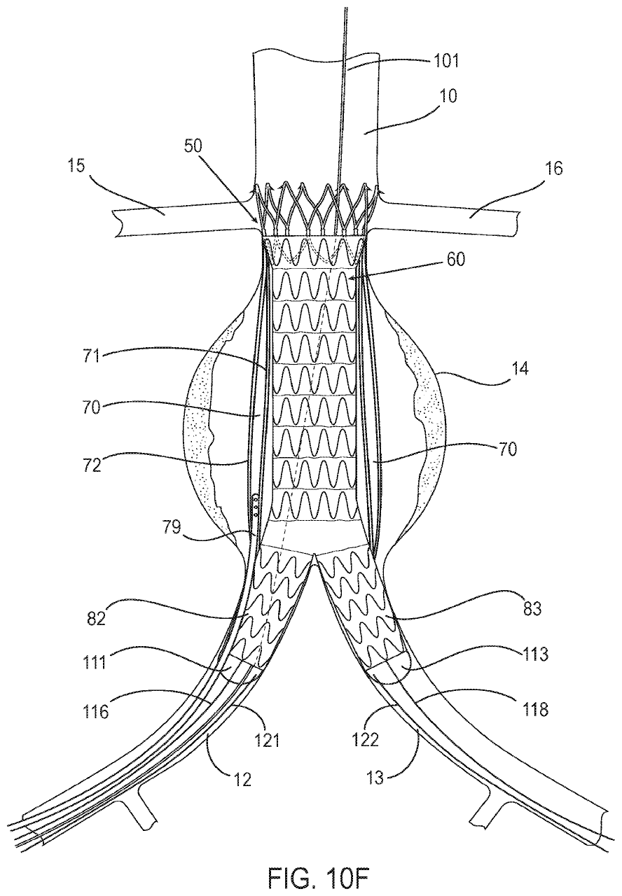

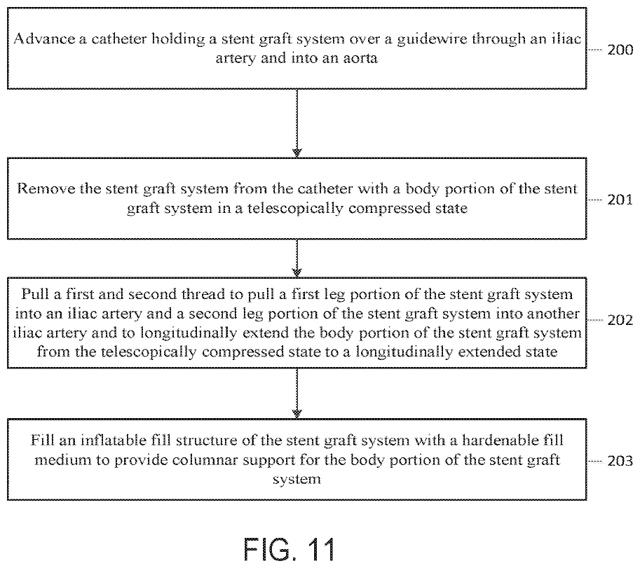

FIGS. 10A, 10B, 10C, 10D, 10E, and 10F are illustrations showing some steps during placement, extension, and filling of the stent graft system 50 in an infrarenal aneurysm, in accordance with various illustrative embodiments. FIG. 11 is a flow diagram illustrating a method of using the stent graft system 50 of FIGS. 10A, 10B, 10C, and 10D, in accordance with an illustrative embodiment. With reference to FIGS, 5, 10A and 11, in step 200 a catheter 102 containing the stent graft system 50 is advanced over a guidewire 101 through the iliac artery 12 and into the aorta 10. With reference to FIGS. 10A, 10B, and 11, in step 201 the stent graft system 50 is removed from the catheter 102 with the body portion 60 in the telescopically compressed state and the inflatable fill structure 70 in an initial state (e.g., relaxed or unstretched state). The body portion 60 can then radially expand, and the barbs 52 of the radially expandable scaffold 51 penetrate into the wall of the aorta 10 to enhance fixation of the stent graft system 50. A fill line 79 is also removed from the catheter 102 and is connected to allow a filling medium (e.g., saline, the hardenable filling medium, and/or the like) to be filled into the inflatable fill structure 70. In various embodiments, the stent graft system 50 also comes in the catheter 102 with a first balloon 111 within the first leg portion 82 and a second balloon 113 within the second leg portion 83. A first fill line 121 and a second fill line 122 are connected to the first balloon 111 and the second balloon 113, respectively, to allow for filling and/or unfilling the first balloon 111 and the second balloon 113. In various embodiments, each of the first balloon 111 and the second balloon 113 may initially be unfilled or partially filled, and may be filled via the first fill line 121 and the second fill line 122, respectively, after the first leg portion 82 and the second leg portion 83 are pulled into the iliac artery 12 and iliac artery 13, respectively.

In various embodiments, there may be a thread 112 attached to the first balloon 111 and a thread 114 attached to the second balloon 113 as shown in FIGS. 10B and 10C. The first thread 112 extends through the iliac artery 12, and the second thread 114 extends through the iliac artery 13. With reference to FIGS. 10C and 11, in step 202 the first thread 112 and the second thread 114 are pulled to pull the first leg portion 82 into the iliac artery 12 and the second leg portion 83 into the iliac artery 13, respectively, and to cause the body portion 60 to longitudinally extend from the compressed state (as in FIG. 10B) to the longitudinally extended state (as in FIG. 10C). As the body portion 60 is longitudinally extended, the inflatable fill structure 70 is longitudinally expanded or extended (e.g., stretched or pulled) in an amount corresponding to an amount that the body portion 60 is longitudinally extended (as in FIG. 10C). However, the present invention is not limited thereto, and in other embodiments, the first thread 1.12 and the second thread 11.4 may be omitted. In this case, the first fill line 121 and the second fill line 122 may be pulled to pull the first leg portion 82 into the iliac artery 12 and the second leg portion 83 into the iliac artery 13.

In various other embodiments, such as the embodiment in FIGS. 10E and 10F, a first wire 116 is connected to the first leg portion 82 and a second wire 118 is connected to the second leg portion 83. The first wire 116 extends through the iliac artery 12, and the second wire 118 extends through the iliac artery 13. In this case, the first wire 116 and the second wire 118 may be pulled to pull the first leg portion 82 into the iliac artery 12 and the second leg portion 83 into iliac artery 13, respectively, and to cause the body portion 60 to longitudinally extend from the compressed state (as in FIG. 10E) to the longitudinally extended state (as in FIG. 10F). As the body portion 60 is longitudinally extended, the inflatable fill structure 70 is longitudinally expanded or extended (e.g., stretched or pulled) in an amount corresponding to an amount that the body portion 60 is longitudinally extended (as in FIG. 10F). The first wire 116 and second wire 118 can then be detached and removed.

Once the first leg portion 82 and the second leg portion 83 are positioned in the iliac artery 12 and the iliac artery 13, respectively, the first balloon 111 and the second balloon 113 may be filled via the first fill line 121 and the second fill line 122, respectively, to radially expand the first leg portion 82 and the second leg portion 83. After filling the first balloon 111 and the second balloon 113, they can be deflated and removed along with the first fill line 121 and the second fill line 122. The first balloon 111 and the second balloon 113 may be filled either before the inflatable fill structure 70 is filled, or alter the inflatable fill structure 70 is filled and the fill line 79 is removed therefrom. However, the present invention is not limited thereto, and in other embodiments, the first balloon 111 and the second balloon 113 may be omitted. In this case, each of the first leg portion 82 and the second leg portion 83 may be radially self-expandable to be expanded after being positioned in the iliac artery 12 and the iliac artery 13 via the first wire 116 and the second wire 118, respectively.

With reference to FIGS. 10D and 11, in step 203, the inflatable fill structure 70 of the stent graft system 50 is filled with a hardenable fill medium 74 through the fill line 79. FIG. 10D shows the stent graft system 50 with the inflatable fill structure 70 filled with the hardenable fill medium 74. In various embodiments, the hardenable fill medium 74 may include, for example, a liquid polymer that hardens as it is dried or cured, such as a hydrogel or the like. However, the present invention is not limited thereto, and any suitable hardenable fill medium may be used to fill the inflatable fill structure 70. In various embodiments, the inflatable fill structure 70 may first be filled with saline to cause expansion of the inflatable fill structure 70 (e.g., longitudinally and/or radially) with the saline then being removed, and then filled with the hardenable fill medium 74, but the present invention is not limited thereto.

The fill tube 79 and guidewire 101, as well as the first and second balloons 111 and 113, the first and second fill lines 121 and 122, and the first and second threads 112 and 114 (refer to FIG. 10C) can then be removed. When the first and second balloons 111 and 113 are filled and/or removed (e.g., via the first and second threads 112 and 114 or the first and second fill lines 121 and 122), the first leg portion 82 and the second leg portion 83 radially expand to contact walls of the iliac arteries 12 and 13, respectively. When inflated or filled as in FIG. 10D, the inflatable fill structure 70 provides columnar support for the body portion 60 of the stent grail system 50.

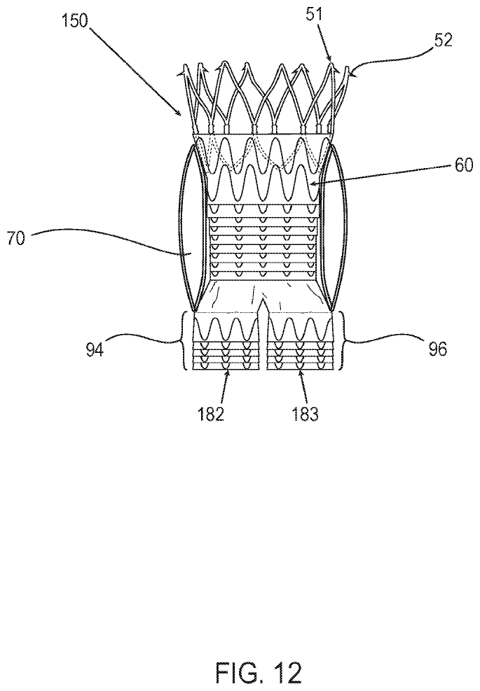

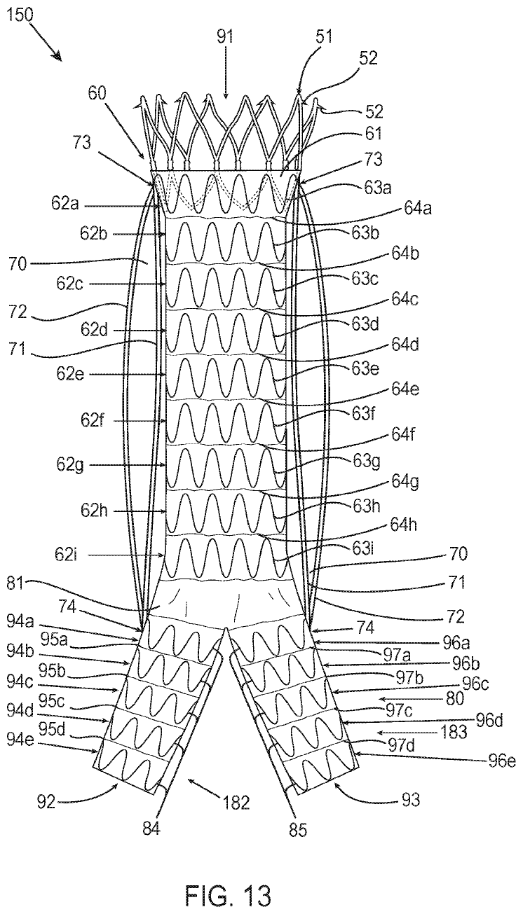

FIG. 12 is an illustration of a stem graft system in accordance with another embodiment in a telescopically compressed state, and FIG. 13 is an illustration of the stent graft system of FIG. 12 in a longitudinally extended state. In FIGS. 12 and 13, the components and elements that are the same or substantially the same as those of the previous embodiments are denoted by like reference numbers, and thus, repeat description may be omitted.

Referring to FIGS. 12 and 13, the first and second leg portions 182 and 183 of a stent graft system 150 include first and second pleated portions 94 and 96, respectively. The first pleated portion 94 includes pleated sections 94a, 94b, 94c, 94d, and 94e, that are divided by pleats 95a, 95b, 95c, and 95d, respectively. The second pleated portion 96 includes pleated sections 96a, 96b, 96c, 96d, and 96e, that are divided by pleats 97a, 97b, 97c, and 97d, respectively. In the telescopically compressed state, the pleated section 94b can fold at least partially up under the pleated section 94a, the pleated section 94c can fold at least partially up under the pleated section 94b, the pleated section 94d can fold at least partially up under the pleated section 94c, and the pleated section 94e can fold at least partially up under the pleated section 94d. Similarly, the pleated section 96b can fold at least partially up under the pleated section 96a, the pleated section 96c can fold at least partially up under the pleated section 96b, the pleated section 96d can fold at least partially up under the pleated section 96c, and the pleated section 96e can fold at least partially up under the pleated section 96d.

In the compressed state, each of the pleated sections may have a slightly smaller diameter than the above adjacent pleated section so that they can fit within each other in a telescoping manner, and when the pleated sections are extended (e.g., fully or partially) in the longitudinal direction, their diameter may expand radially. In various embodiments, each of the first and second pleated portions 94 and 96 may be formed such that the crown valleys are tucked abluminally, adluminally, or having one end tucked abluminally while the other end is tucked adluminally. is some embodiments, the first and second pleated portions 94 and 96 may have different tucking arrangements from each other. For example, the crown valleys of the first pleated portion 94 may be tucked abluminally, while the crown valleys of the section pleated portion 96 are tucked adluminally, or vice versa.

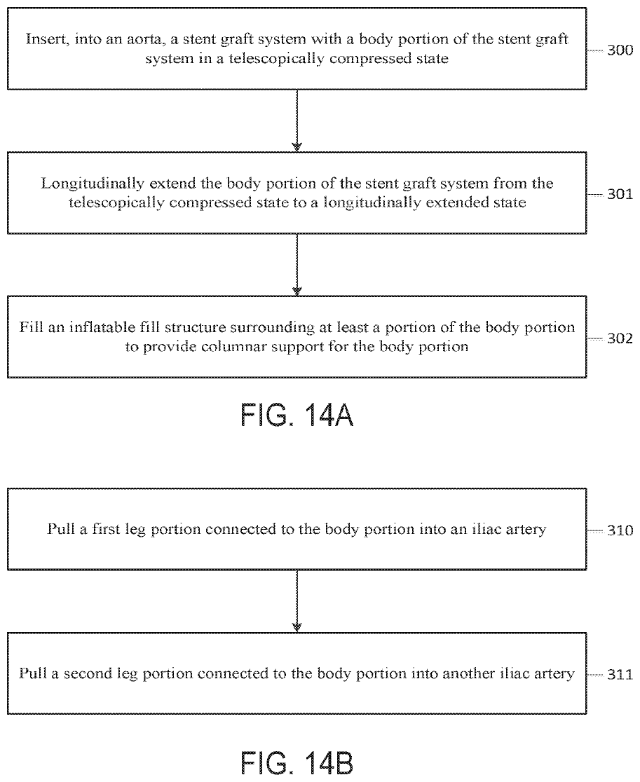

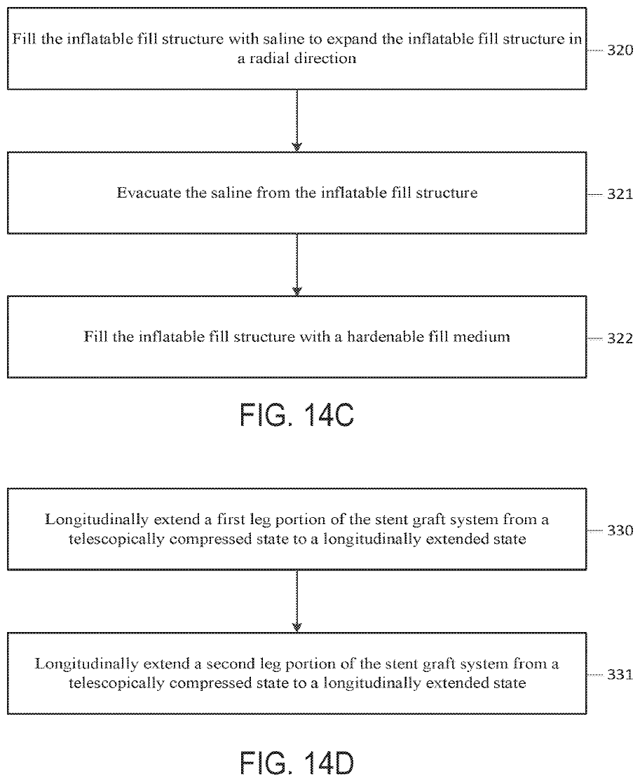

FIG. 14A illustrates a flowchart of a method in accordance with various embodiments for deploying a stent graft system to repair an aneurysm. In step 300, the stent graft system is inserted into an aorta with a body portion of the stent graft system in a telescopically compressed state. In step 301, the body portion of the stent graft system is longitudinally extended from the telescopically compressed state to a longitudinally extended state. In step 302, an inflatable fill structure surrounding at least a portion of the body portion is filled to provide columnar support for the body portion.

In various embodiments, the inflatable fill structure is attached to at least a top of the body portion. In various embodiments, the inflatable fill structure is not attached at a central part of the body portion. In some embodiments, the inflatable fill structure expands in a longitudinal direction as the body portion is extended in the longitudinal direction. Also, in some embodiments, an amount that the inflatable fill structure expands in the longitudinal direction corresponds to an amount that the body portion extends in the longitudinal direction.

FIG. 14B illustrates a method in accordance with an embodiment for longitudinally extending a body portion of a stent graft system. In step 310, a first leg portion of the stent graft system that is connected to the body portion is pulled into an iliac artery. In step 311, a second leg portion of the stent graft system that is connected to the body portion is pulled into another iliac artery. In various embodiments, the steps 310 and 311 are performed at a same time.

FIG. 14C illustrates a method in accordance with an embodiment for filling an inflatable fill structure. In step 320, the inflatable fill structure is filled with saline to expand the inflatable fill structure in a radial direction. In step 321, the saline is evacuated from the inflatable fill structure. In step 322, the inflatable fill structure is filled with a hardenable till medium. In various embodiments, the hardenable fill medium comprises a polymer. In various embodiments, the inflatable fill structure is radially expanded to conform to an inner surface of an aorta after being longitudinally extended along with the body portion.

FIG. 14D illustrates a flowchart of a method in accordance with an embodiment that can be used with the method of FIG. 14A. With reference to FIG. 14D, in step 330 a first leg portion of the stent graft system is longitudinally extended from a telescopically compressed state to a longitudinally extended state. In step 331, a second leg portion of the stent graft system is longitudinally extended from a telescopically compressed state to a longitudinally extended state. In various embodiments, the steps 330 and 331 are performed at a same time, In various embodiments, the first leg portion and the second leg portion are connected to the body portion of the stent graft system.

According to various embodiments, a stent graft system includes a body portion that is longitudinally extendable from a telescopically compressed state, and includes one or more inflatable fill structures surrounding the body portion. In various embodiments, an inflatable fill structure surrounding the body portion is highly conformal, and may be longitudinally expanded (e.g., stretched or pulled) from an initial state (e.g., rested or unstretched state), as the body portion is longitudinally extended. The amount that the inflatable fill structure is longitudinally expanded may correspond to an amount that the body portion is longitudinally extended.

According to various embodiments, the stent graft system may further include a plurality of leg portions connected to the body portion. One or more of the leg portions may be longitudinally extendable from a compressed state.

Accordingly, various stent graft systems that can be used in patients with differing aneurysm lengths and volumes have been described. The stent graft systems according to various embodiments can conform to various dimensions of an aneurysm being treated, and thus, allow for treating a wide range of patients.

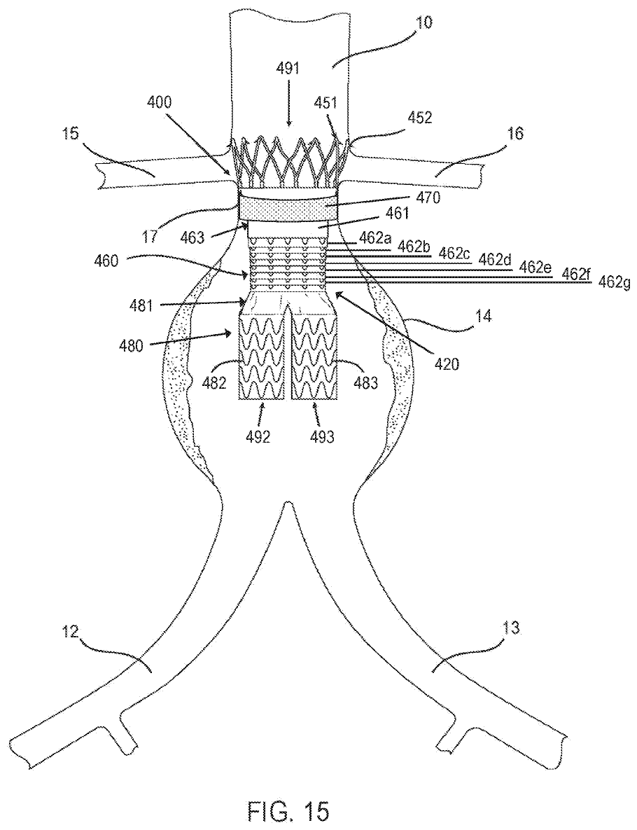

FIG. 15 illustrates a stent graft system 400 in accordance with an embodiment within the aorta 10. The stent graft system 400 includes a stent graft 420, a radially expandable scaffold 451, and an inflatable fill structure 470. The stent graft 420 includes a body portion 460 with a plurality of pleated sections 462a, 462b, 462c, 462d, 462e, 462f, 462g that are configured to be extended from a telescopically compressed state to a longitudinally extended state. The radially expandable scaffold 451 is attached to a top of the body portion 460 and has one or more fixation elements 452. for penetrating into an aortic wall. The inflatable fill structure 470 is positioned at top section 463 of the body portion 460 and is configured to not expand in a longitudinal direction as the body portion 460 is extended in the longitudinal direction. In various embodiments, the inflatable fill structure 470 is configured to provide a seal at the proximal neck 17 of an aneurysm defined by the aneurysm sac 14.

The stent graft 420 includes the body portion 460 and a bifurcation portion 480. The bifurcation portion 480 includes a transition portion 481, a first leg portion 482, and a second leg portion 483. The stem graft 420 includes a graft 461. In various embodiments, the graft 461 is used for the body portion 460 and the bifurcation portion 480. In some embodiments, one or more other grafts may be used for each of the portions of the stem graft 420. In various embodiments, the graft 461 is made of a polymer. in some embodiments, the graft 461 is made of expanded Polytetrafluoroethylene (ePTFE). The body portion 460 includes the pleated sections 462a, 462b, 462c, 462d, 462e, 462f, and 462g. Each of the pleated sections 462a, 462b, 462c, 462d, 462e, 462f, and 462g includes a scaffold, respectively, that is encapsulated within or attached to a corresponding portion of the graft 461. The scaffolds of the pleated sections 462a, 462b, 462c, 462d, 462e, 462f, and 462g may each comprise, for example, a sinusoidal stent frame in a ring shape and may be made from, for example, cobalt-chromium (CoCr) alloy, stainless steel, nitinol, or the like.

Each of the pleated sections 462a, 462b, 462c, 462d, 462e, 462f, and 462g ends with a pleat, respectively, in the graft 461. The pleats in the graft 461 allow for the graft 461 to fold in at those locations, so that the body portion 460 can be telescoping to be in a compressed state, and to allow for movement from the compressed state to the longitudinally extended state. In various embodiments, a length of the body portion 460 in the compressed state may be less than one-fourth of the length of the body portion 460 in the extended state. In various embodiments, a length of the body portion 460 in the compressed state may be less than one-hall of the length of the body portion 460 in the extended state. When extended, the portion of the graft 461 for the body portion 460 is a generally tubular shape that allows for blood flow through a lumen within the body portion 460. The stent graft system 400 includes the radially expandable scaffold 451 connected to the top of the body portion 460 for providing fixation of the stem graft system 400 in the aorta 10 along an aortic wall. The radially expandable scaffold 451 includes the one or more fixation elements 452 that may, for example, be hooks or barbs for penetrating into an aortic wall and thereby enhancing fixation. In various embodiments, the one or more fixation elements 452 are configured to attach to the aortic wall above the renal arteries 15 and 16.

The transition portion 481 includes a portion of the graft 461 that extends from the body portion 460 to each of the first leg portion 482 and the second leg portion 483. The transition portion 481 may be made of the same or different material from that of the graft 461. For example, in various embodiments, the transition portion 481 may be made of, for example, Teflon. In various embodiments, the first leg portion 482 includes radially expandable scaffolds that are encapsulated within or attached to a corresponding portion of the graft 461. Also, in various embodiments, the second leg portion 483 includes radially expandable scaffolds that are encapsulated within or attached to a corresponding portion of the graft 461. Each of the first leg portion 482 and the second leg portion 483 is a generally tubular shape that allows for blood flow through a lumen within the first leg portion 482 and the second leg portion 483, respectively. The stent graft system 400 has a proximal end 491 for receiving blood flow and distal ends 492 and 493 out of which the blood is able to flow.

The stein graft system 400 includes the inflatable fill structure 470. The inflatable fill structure 470 may surround (e.g., entirely surround) the outer circumference of the top section 463 of the body portion 460, and may be a single inflatable fill structure or a plurality of inflatable fill structures arranged around the body portion 460. In some embodiments, the inflatable fill structure 470 is located between layers of the graft 461. In some embodiments, the inflatable fill structure 470 is an endobag or the like. In some embodiments, the inflatable fill structure 470 is attached to the top section 463 of the body portion 460. In various embodiments, the inflatable fill structure 470 is fillable through a removable fill tube with a hardenable filling material such as Polyethylene glycol (PEG) or another polymer that may be polymerized in situ.

The stent graft system 400 is extendable to extend from a telescopically compressed state to a longitudinally extended state in the aorta 10. In the longitudinally extended state the first leg portion 482 extends into the iliac artery 12 and the second leg portion 483 extends into the iliac artery 13. In various embodiments, the body portion 460 of the stent graft system 400 is able to be extended and/or expand across the aneurysm sac 14 to exclude the aneurysm sac 14 from aortic blood pressure. The stent graft system 400 includes the body portion 460 that can be placed in the aorta 10, and the first and second leg portions 482 and 483 extending from the body portion 460 that can be placed into the iliac arteries 12 and 13, respectively. The inflatable fill structure 470 can be filled to press against a wall of the aorta 10 at the proximal neck 17 to create a proximal seal. The distal ends 492 and 493 of the first and second leg portions 482 and 483 can radially expand against walls of the iliac arteries 12 and 13, respectively, to form distal seals.

The barbs 452 of the radially expandable scaffold 451 can penetrate into the wall of the aorta 10, thereby enhancing fixation of the stent graft system 400. Blood is able to flow from the proximal end 491 through the body portion 460 and out of the distal ends 492 arid 493 of the first and second leg portions 482 and 483, respectively. The inflatable fill structure 470 is initially in an uninflated state, but is tillable with a fill medium. In various embodiments, the inflatable fill structure 470 is located entirely above the pleated sections 462a, 462b, 462c, 462d, 462e, 462f, and 462g of the body portion 460 such that when the pleated sections 462a, 462b, 462c, 462d, 462e, 462f, and 462g of the body portion 460 are pulled from a telescopically compressed state to a longitudinally extended state the inflatable fill structure 470 is not expanded in a longitudinal direction and remains in place. In some embodiments, the inflatable fill structure 470 is filled with a hardenable fill medium to create a seal against the proximal neck 17 prior to the pleated sections 462a, 462b, 462c, 462d, 462e, 462f, and 462g of the body portion 460 being pulled from a telescopically compressed state to a longitudinally extended state. In some embodiments, the inflatable fill structure 470 is filled with a hardenable fill medium to create a seal against the proximal neck 17 after to the pleated sections 462a, 462b, 462c, 462d, 462e, 462f, and 462g of the body portion 460 have been pulled from a telescopically compressed state to a longitudinally extended state.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. Various modifications and changes that come within the meaning and range of equivalency of the claims are intended to be within the scope of the invention. Thus, while certain embodiments of the present invention have been illustrated and described, it is understood by those of ordinary skill in the art that certain modifications and changes can be made to the described embodiments without departing from the spirit and scope of the present invention.

What is claimed is:

1. A stent graft system, comprising:
a stern graft including a body portion with a plurality of pleated sections that are configured to be extended from a telescopically compressed state to a longitudinally extended state;
a radially expandable scaffold attached to a top of the body portion, and haying one or more fixation elements for penetrating into an aortic wall; and
an inflatable fill structure attached at a top of the body portion and configured to expand in a longitudinal direction as the body portion is extended in the longitudinal direction.

2. The stent graft system of claim 1,
wherein the inflatable fill structure is not attached at a central part of the body portion.

3. The stem graft system of claim 2, wherein the inflatable fill structure is further attached at a lower part of the body portion.

4. The stent graft system of claim 1, wherein an amount that the inflatable fill structure expands in the longitudinal direction corresponds to an amount that the body portion is extended in the longitudinal direction.

5. The stent graft system of claim 1, wherein the inflatable structure comprises:
an inner wall adjacent to an outer surface of the body portion; and
an outer wall.

6. The stent graft system of claim 5, wherein the inner wall is configured to contact the outer surface of the body portion when the inflatable fill structure is inflated to provide columnar support to the body portion.

7. The stent graft system of claim 6, wherein the outer wall is configured to conform to an inner surface of a vessel in which the stent graft is inserted.

8. The stent graft system of claim 1, wherein the stent graft further includes:
a first leg portion;
a second leg portion; and
a transition portion connecting the first leg portion and the second leg portion to the body portion.

9. The stent graft system of claim 8, wherein at least one of the first leg portion and the second leg portion is configured to be extendable from a telescopically compressed state to a longitudinally extended state.

10. The stent graft system of claim 1, wherein a length of the body portion in the telescopically compressed state is less than one-fourth of a length of the body portion in the longitudinally extended state.

11. A method for deploying a stent graft system to repair an aneurysm, comprising:
inserting, into an aorta, the stent graft system with a body portion of the stent graft system in a telescopically compressed state;
longitudinally extending the body portion of the stent graft system from the telescopically compressed state to a longitudinally extended state; and
filling an inflatable fill structure surrounding at least a portion of the body portion to provide columnar support for the body portion.

12. The method of claim 11, wherein the inflatable fill structure is attached to at least a top of the body portion.

13. The method of claim 12, wherein the inflatable fill structure is not attached at a central part of the body portion.

14. The method of claim 11, wherein the inflatable fill structure expands in a longitudinal direction as the body portion is extended in the longitudinal direction.

15. The method of claim 14, wherein an amount that the inflatable fill structure expands in the longitudinal direction corresponds to an amount that the body portion extends in the longitudinal direction.

16. The method of claim 11, wherein the longitudinally extending of the body portion comprises:
pulling a first leg portion connected to the body portion into an iliac artery; and
pulling a second leg portion connected to the body portion into another iliac artery.

17. The method of claim 11, wherein the filling of the inflatable fill structure comprises:
filling the inflatable fill structure with saline to expand the inflatable fill structure in a radial direction;
evacuating the saline from the inflatable fill structure; and
filling the inflatable till structure with a hardenable fill medium.

18. The method of claim 17, wherein the hardenable fill medium comprises a polymer.

19. The method of claim 11, wherein the inflatable fill structure is radially expanded to conform to an inner surface of the aorta after being longitudinally extended along with the body portion.

20. The method of claim 11, further comprising:
longitudinally extending a first leg portion of the stent graft system from a telescopically compressed state to a longitudinally extended state; and
longitudinally extending a second leg portion of the stent graft system from a telescopically compressed state to a longitudinally extended state;
wherein the first leg portion and the second leg portion are connected to the body portion of the stent graft system.

21. A stent graft system, comprising:
a stent graft including a body portion with a plurality of pleated sections that are configured to be extended from a telescopically compressed state to a longitudinally extended state;
a radially expandable scaffold attached to a top of the body portion, and having one or more fixation elements for penetrating into an aortic wall; and
an inflatable fill structure positioned at top section of the body portion and configured to not expand in a longitudinal direction as the body portion is extended in the longitudinal direction.

22. The stem graft system of claim 21,
wherein the inflatable fill structure is configured to provide a seal at a proximal neck of an aneurysm.
